(19) United States
(12) Patent Application Publication
(10) Pub. No.: US 2024/0145078 A1
(43) Pub. Date: May 2, 2024

(54) Improved Nurse Rounding Detection

(71) Applicant: Sanford Health, Sioux Falls, SD (US)

(72) Inventors: Heather Perez, Sioux Falls, SD (US); Merideth Bell, Sioux Falls, SD (US); Jason Thomas, Sioux Falls, SD (US); Jordan Mudek, Sioux Falls, SD (US); James Engelstad, Sioux Falls, SD (US)

(21) Appl. No.: 18/548,005

(22) PCT Filed: Feb. 26, 2022
(86) PCT No.: PCT/US22/18050

§ 371 Date: Aug. 25, 2023

Related U.S. Application Data
(60) Provisional application No. 63/154,683, filed on Feb. 27, 2021.

(51) Int. Cl. G16H 40/20; G16H 40/67

(52) U.S. Cl. CPC G16H 40/20; G16H 40/67

(57) ABSTRACT

Embodiments provided herein facilitate the monitoring and management of regular healthcare tasks for patients in a healthcare system, e.g., “nurse rounding” tasks. These embodiments include providing a button or other activator that is associated with a patient and a timer that is associated with the patient and that represents the time since a rounding task was last performed for the patient. Activation of the activator by a nurse or other healthcare provider causes the patient's timer to reset. The status of the timers for a number of patients can be provided on a display, e.g., at a nurse station, to allow nurses or other healthcare providers to know at a glance which patients are currently, or likely to soon become, in need of another rounding care visit. The activator output can also be automatically registered in order to monitor performance of rounding tasks for the patient over time.

### CROSS-REFERENCE TO RELATED APPLICATION

The present application is a non-provisional patent application claiming priority to U.S. Provisional Patent Application No. 63/154,683, filed on Feb. 27, 2021, the contents of which are hereby incorporated by reference.

### BACKGROUND

In a hospital or other healthcare environment, a variety of tasks must be performed for each patient according to a set schedule and/or at a rate more frequent than some specified interval in order to provide superior patient care. Such patient care tasks can include assisting or otherwise attending to the patient's need to urinate/defecate (whether in their bed or by assisting the patient to move to a toilet), assessing and/or taking steps to manage the patient's pain, making certain that the patient has access to their personal effects and/or assisting them in accessing or putting away such items, or positioning/repositioning the patient (e.g., to reduce the incidence of pressure ulcers, to accommodate drainage or other post-surgical considerations).

It can be difficult to monitor the performance of such tasks and to motivate their completion according to a set schedule/rate. For example, nurses or other healthcare providers could be required to make paper logs of their completion of such tasks (e.g., logging time, patient ID, task(s) performed, pertinent information about the task/patient); however this requires additional caregiver time to complete and can be expensive and difficult to accurately digitize. In another example, the healthcare providers could be required to log in to health records platform (e.g., via a phone or tablet); however, this can also be a time-intensive process, especially where two-factor authentication or other means for protecting patient privacy are enforced in order to access the health records platform. These existing logging methods are expensive with respect to healthcare provider time and effort and often result in incomplete or imperfect records. Moreover, while such methods may facilitate monitoring of the performance of such regular patient care tasks, they do not provide means for alerting healthcare providers to the fact that individual patients may, at a particular time, require such tasks to be performed and/or that the performance of such tasks for a patient may be overdue.

### SUMMARY

A first example embodiment may involve a system that includes: (i) an activator; (ii) a display; and (iii) a controller that includes one or more processors. The one or more processors are configured to perform controller operations including: (a) indicating, via the display, that a timer associated with a particular patient has exceeded a threshold duration; (b) associating the activator with the particular patient; (c) detecting that the activator has been activated; (d) responsive to detecting that the activator has been activated, resetting the timer; and (e) updating an indication on the display to reflect that the timer has been reset.

A second example embodiment may involve a method that includes: (i) indicating, via a display, that a timer associated with a particular patient has exceeded a threshold duration; (ii) associating an activator with the particular patient; (iii) detecting that the activator has been activated; (iv) responsive to detecting that the activator has been activated, resetting the timer; and (v) updating an indication on the display to reflect that the timer has been reset.

In a third example embodiment, an article of manufacture may include a transitory or non-transitory computer-readable medium, having stored thereon program instructions that, upon execution by a computing system, cause the computing system to perform operations in accordance with the second example embodiment.

In a fourth example embodiment, a computing system may include at least one processor, as well as memory and program instructions. The program instructions may be stored in the memory, and upon execution by the at least one processor, cause the computing system to perform operations in accordance with the second example embodiment.

In a fifth example embodiment, a system may include various means for carrying out each of the operations of the second example embodiment.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates an example healthcare environment, in accordance with example embodiments.

FIG. 2 is a flow chart, in accordance with example embodiments.

FIG. 3 depicts elements of an example system, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. Overview

As used herein, the singular forms “a”, “an” and “the” include plural referents unless the context clearly dictates otherwise. “And” as used herein is interchangeably used with “or” unless expressly stated otherwise.

All embodiments of any aspect of the disclosure can be used in combination, unless the context clearly dictates otherwise.

“Care rounding,” sometimes referred to as “nurse rounding” or “hourly nurse rounds,” refers to a process of periodically visiting patients to ensure that they are comfortable, to take care of needs or concerns they may have, to check in on their physical or mental state, and/or to perform additional or alternative tasks on a regular basis (e.g., once every two hours for a particular patient, or hourly from 6 AM to 10 PM and every two hours otherwise for a particular patient). Implementing such a rounding process in a hospital setting has been shown to result in improved patient outcomes, including a 50% reduction in falls, decreased use of the call button, a 30% decrease in the rate of nurse alert events, and increased communication between patients and healthcare staff.

However, practical implementation of regular nurse rounding can be difficult. In practice, nurses have a large number of concurrent duties and concerns, so it can be difficult for them to perform rounding according to a set threshold rate. This difficulty stems from having to find the time to perform these rounds as well as the additional time and effort necessary to either (1) maintain a record or memory of the timing of those rounds or (2) to create and comply with a set rounding schedule, which itself can be easily thrown off by the realities of ongoing patient care and tending to patient needs. These practicalities also make manual logging or other methods for recording compliance with rounding requirements difficult, further reducing compliance and increasing nurse workload. Paper logs may be posted near patients (e.g., near the door to a patient's room) for nurses to record the times at which they round; however, the time required to initially log such events, as well as the increased cost of transcribing the logs, can result in such logs being inaccurate due to noncompliance or transcription errors. The use of conventional electronic logging, which may require nurses to engage in multi-factor logins many additional times per day to protect sensitive patient data, presents similar issues of noncompliance, inaccuracy, and increased nurse workload.

The embodiments described herein provide technical solutions to these concerns, allowing for the implementation of nurse rounding in a manner that provides easy-to-check reminders of patient rounding needs as well and low-effort, unobtrusive reporting of completed rounding events. These embodiments include providing a “rounding button” near the patient (e.g., near the door to a patient's room) that a nurse can easily and quickly interact with to indicate that he/she has completed a rounding event with the patient. Activating the “rounding button” (which can include a button, a lever, a touch panel, or some other variety of activator that a nurse or other healthcare provider can easily and quickly press or otherwise activate) can then result in a rounding timer for the patient being reset.

The status of the timers of a set of patients (e.g., all the patients on a floor, all the patients under a particular nurse's care, all the patients on a floor whose timers indicate that they should receive rounding soon) can be provided on a display to quickly and easily indicate to a nurse or set of nurses when a patient is in need of rounding. Such a display could be a dedicated display (e.g., a screen provided at a nurse station and visible from a large portion of a patient care floor, or an indicator light outside each patient's room) or a display used periodically and/or upon request to indicate the state of one or more patient's rounding needs (e.g., a display of a tablet or other device used by a nurse and having installed thereon an app or other program to access and/or maintain rounding timers for patients and to display information related thereto).

These embodiments allow a nurse or other healthcare professional to quickly and easily indicate that they have completed a round or other intentional care event(s) with a patient (or some other healthcare event or task) without requiring them to log in to yet another electronic system, to take time to manually record the time and other parameters of such a visit, and/or to engage in some other effortful task. This functionality is facilitated in part by associating each “rounding button” or other variety of activator with a particular patient so that pressing or otherwise activating the activator results in resetting or otherwise modifying a rounding timer for that particular patient. Thus, the nurse does not need to manually record or otherwise indicate the identity of the patient, allowing the nurse to forego engaging in yet another secured system login. This solution also allows for technological systems to passively identify attributes of the round like patient identity, career identity, room number, duration, and timing without additional effort on the part of the career. The association between a particular activator (e.g., button) and a particular patient may be accomplished in a variety of ways, as described below.

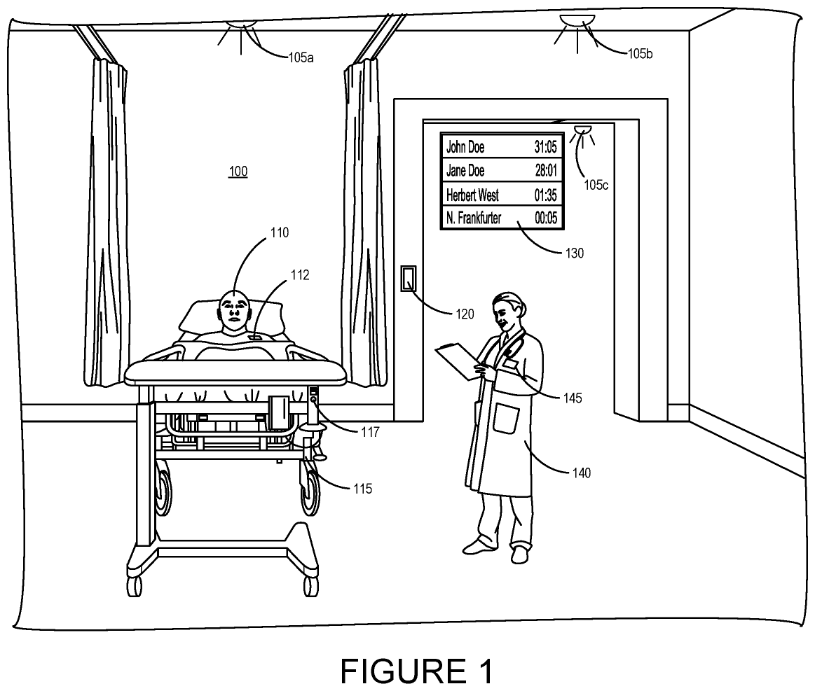

FIG. 1 illustrates, by way of example, aspects of the systems and methods described herein to facilitate “nurse rounding” or other repeated care tasks. A patient 110 is resting in a hospital bed 115 in their room 100 (which may be shared with one or more other patient(s), not shown). A display 130 provides an indication that the patient 110 is due for a rounding or will be due soon for a rounding visit. Accordingly, a nurse 140 or other healthcare provider can go to the patient 110 and perform the activities (e.g., asking the patient about their concerns, needs, symptoms, state of mind, etc., refreshing refreshments, checking vital signs of the patient, checking the operation of lines or other medical interventions or equipment, checking bandages or wound sites) that are part of a rounding event. After completing these rounding tasks, the nurse 140 can activate an activator 120 (e.g., by pressing a button of the activator 120) to indicate that they have rounded with the patient 110. This results in the timer for the patient being reset, reduced, or otherwise modified. This change in the patient's rounding timer can then be reflected, in real time or near real time, on the display 130 (e.g., by changing a color of the indication for the patient 110, by showing a change in the numerical value of the timer for the patient 110, by removing the indication of the patient's name or room number from the display 130 until they are due for rounding again, etc.). Also depicted in FIG. 1 are a locator device 145 worn or carried by the nurse 140, a locator device 112 worn or carried by the patient 110, a locator device 117 that is part of and/or mounted to the hospital bed 115, and emitter devices 105a, 105b, 105c that may be included and used to provide other functionality described below.

Such a system for facilitating and monitoring rounding (or other repeated healthcare tasks) may be technically implemented in a variety of ways. In some examples, the activator 120, display 130, and/or other devices (e.g., 112, 117, 145, 105a-c) may be in communication with a central controller (e.g., via WiFi, wired connections, Bluetooth, etc.) that maintains timers for patients and that implements other aspects of the systems and methods described herein. Such a controller may be physically co-located in a hospital in which the patient 110 is housed or may be off-site (e.g., part of a server farm, part of a healthcare system administrative office complex).

Such a controller may be devoted specifically to implementing some or all of the functionality described herein. For example, a server may be configured to communicate with displays, activators, locator devices, and other components to locate patients and healthcare providers, implement and maintain rounding timers, provide indications thereof, etc. In another example, a server may communicate with a Real Time Location Services (RTLS) to access information about the locations of healthcare providers, patients, locator devices, etc. and may use this data, in combination with information from activators, displays, etc. to implement the functionality described herein. Alternatively, such a controller may provide a variety of services, while the functionality described herein is implemented as a service, daemon, plugin, app, package, or other hardware and/or software sub-unit thereof. For example, the controller may be provided as part of an RTLS system, and the functionality described herein may be implemented as a downloadable package or program running as part of the RTLS system and using communications, device locating, display, and other built-in functionalities of the RTLS system to implement the described rounding functionality.

Additionally or alternatively, such a system for facilitating and monitoring rounding (or other repeated healthcare tasks) may be technically implemented as an ad-hoc or distributed system. For example, a tablet, smart phone, or other portable computing and communications device associated with a nurse could maintain rounding timers for each patient under the nurse's care and could communicate with activators 120, locator devices 112, 117, positioning systems or resources (e.g., GPS, transmitters 105a-c, etc.), or other systems to implement the functionality described herein. This could be done in a secure manner by implementing cryptographic or otherwise secure communications between the nurse's device and the activator and/or other systems. Additionally or alternatively, this functionality could be accomplished in a manner that protects sensitive patient data by operating without reference to patient data in any of the transmissions/communications. For example, random identifier codes or the like could be transmitted between the portable device and activators 120, locators 112, 117, etc. with the mapping between such identifier codes and patient names or other sensitive patient data being present only on the portable device (e.g., as a result of a nurse manually entering a patient name as part of a pairing process with the activator 120, locator device 112, 117, etc.).

Similarly, the activator 120, display 130, and/or other devices (e.g., 112, 117, 145, 105a-c) may be purpose-built for use with the methods and systems described herein or may be commodity devices configured and/or used to provide the functionality described herein. For example, the activator 120 and locator devices 112, 117, 145 could be commodity devices that are part of a commercial RTLS system, with the functionality described herein implemented as software on a server or other controller of the RTLS system and/or as separate software and/or hardware that is in communication with such an RTLS system.

In order for activation of a button or other activator (e.g., 120) to result in resetting of a rounding timer or other operations related to a particular patient (e.g., 110), the activator must be associated with that particular patient. Such association of activator to patient can be accomplished in a variety of ways. In some examples, some level of manual activity could be involved. For example, a newly-admitted patient could be associated (e.g., in a medical records system) with an identifier or other resource locator indicative of the activator. In some examples, the activator could be associated with a particular room or location within a room (manually or via some other means) and then the patient could be associated with that room.

Associating the activator with the room could include manually associating the activator with the room or using an RTLS or other system to detect the location of the activator and then determining that the detected location corresponds to the room. For example, a microphone, antenna, coil, photodetector, or other sensor element(s) incorporated into a device with the activator could detect an infrared signal, a sound signal (e.g. ultrasound signal), a radio frequency signal (e.g., a WiFi signal, a directional radiolocating signal), or some other signal that is indicative of location(s) within a hospital. For example, the locating signal emitters 105a, 105b, 105c in FIG. 1 could each emit respective differently-coded infrared, sound, radio frequency, or other signals such that a sensor (e.g., in the activator 120 and/or one of the patient locators 112, 117) can detect the emitted wireless signal(s) and use them to determine a location (e.g., by determining that a particular one of the emitted signals is strongest, and thus that the sensor is located proximate to the corresponding one of the locating signal emitters 105a, 105b, 105c). This detected signal could then be used to determine the location of the activator, e.g., by determining that an identifier encoded into the signal corresponds to an identifier code for a particular hospital room.

Associating the patient with a particular room or location could be performed manually or automatically. For example, a medical record system could be accessed to provide an up-to-date reference to the location of a patient based on procedure records, admission records, or other records of patient procedures, movements, transfers, and/or care. Additionally or alternatively, an RTLS or other system could be used to detect the location of the patient. For example, a patient locator could be worn by the patient (e.g., in a wristband) and/or incorporated into a hospital bed or other equipment used by the patient (e.g., an RTLS locator or other locator device incorporated into the bed and/or mounted onto the bed somehow). The location of the patient could be determined and compared to a known location of an activator (e.g., detected as described above) in order to associate the patient with the activator. Additionally or alternatively, sound, infrared, radio frequency, or other wireless signals detected by sensors on the activator and on a patient locator device could be compared in order to determine that the patient and the activator are proximate to each other, thus associating the activator with the patient.

The location of a patient can be used to control the behavior of the activator 120 and/or of the rounding timer associated with the patient. As noted above, such a patient location can be determined via polling a healthcare procedure database or other care record(s), via detection of a patient locator worn by the patient, via detection of a patient locator incorporated into and/or disposed on a hospital bed associated with the patient, or via some other means. In some examples, resetting of the patient's rounding timer as a result of activation of their associated activator may be dependent upon the determined patient location being proximate to (e.g., in the same room as) the activator. This could be done to prevent the patient's rounding timer from being reset by erroneous activation of the activator when they are not present, or after they have been transferred to a different room.

Indeed, the detected presence or absence of a patient in their room or at some other location may be used to pause their rounding timer and/or to reset their rounding timer so that a nurse is not instructed to round them when they are receiving a procedure or otherwise not present. The patient having received particular procedures could result in modification of their rounding timer, e.g., reducing the timer duration after they receive a procedure necessitating more frequent rounding and/or wellness checks. A system for providing this functionality could poll a medical records database to determine that the patient had received such a procedure and thus update the patient's rounding timer properties accordingly. In some examples, the rounding timer for a particular patient could be initialized in response to their being admitted into a healthcare system, as determined by accessing a database that includes such admission records.

As noted above, providing a display that can be easily scanned to determine which patients are in need of rounding, or who will soon need rounding, increases compliance with regular rounding procedures and leads to improved patient outcomes. Such displays may be used by floor nurses, or by nurses assigned to particular patients, to provide rounding and to plan their other tasks such that each patient receives rounding care in a manner that is compatible with a specified rounding frequency while also minimally impacting the nurses' other duties. As depicted in FIG. 1, such a display could be a full visual display that provides the names and timer values for every patient in a set of patients, e.g., every patient on a floor. The patients could be in alphabetical or room number order, or could be ordered according to their accumulated timer values. In some examples, the indication for each patient could be color-coded according to how close to the threshold rounding period their timer is. For example, a patient whose timer has exceeded the rounding duration could be indicated in red, while a patient whose rounding timer has not, but is getting close (e.g., has surpassed a second, lesser threshold) could be indicated in yellow. Other colors are possible. In some examples, patients may be removed from such a display when their timers are reset, only to be reintroduced when their timers exceed a threshold duration (e.g., the desired rounding period, or some lesser threshold so as to provide advance indication of patients who are about to require rounding).

As illustrated in FIG. 1, a single display 130 is provided that provides textual indications of the identities and timer values of patients. However, this is intended as a non-limiting example embodiment of a display that can provide indications related to the state of one or more patients' rounding timers. For example, more than one such display could be provided, e.g., two displays, one located at each of two stations on a patient care floor. Additionally or alternatively, such a display could be provided as part of a portable device, e.g., as an informational screen or app on a tablet, smart phone, or other portable computing and communications device held or worn by a nurse or other healthcare provider. In some examples, the display could be provided as a simple color-coded lamp on the outside of each patient's room, being lit to indicate that the patient inside is in need of rounding or will be soon. In some examples, audio alerts could also be provided.

In some examples, the location of one or more nurses (e.g., of a nurse specifically assigned to perform rounding for a particular patient) could be detected and used to inform the operation of a rounding system as described herein. For example, the resetting of a patient's timer in response to activation of an activator could be contingent upon a specified nurse, or one of a set of specified nurses, being proximate to the activator (in addition to potentially other factors, e.g., the presence of the patient in the room proximate to the activator, as described above). Indeed, the resetting of the patient's rounding timer could be contingent on the nurse having been present proximate to the activator and/or the patient for more than a threshold period of time. This could be done to ensure that the nurse has actually taken the time necessary to perform the assigned rounding tasks and/or to prevent erroneous resetting of the patient's timer due to erroneous activation of the activator. Determining that a nurse is proximate to the patient and/or activator could include using an RTLS or other system to detect the location of the nurse and then to compare that determined location to a known location of the activator and/or patient. For example, sound, infrared, radio frequency, or other wireless signals detected by sensors on the activator and on a nurse locator device worn or carried by the nurse could be compared in order to determine that the nurse and the activator are proximate to each other.

The systems and method described herein can improve patient care by facilitating the consistent rounding of patients and/or the performance of other repeated care tasks according to a specified frequency. However, these systems and methods can also improve patient outcomes by enabling the logging of accurate data about such rounding or other healthcare tasks in a cost-effective and low-effort manner, allowing such data to inform improvements in healthcare planning and provision. For example, the timing of each round received by a patient (as indicated by activation of the activator associated with the patient) could be recorded and used to determine whether the patient received the prescribed amount of rounding care, and further whether any changes in the patient's health outcomes might be related to the timing of rounding visits received by the patient. Where nurse locations are also tracked in relation to activation of the activator, the identity of the rounding nurse can also be recorded and used to analyze patient care and/or to analyze the performance of individual nurses or other healthcare providers.

Such data could be recorded in, compatible with, integrated into, or otherwise associated with an electronic health records (EHR) system, e.g., Epic™. Additionally or alternatively, aspects of the methods described herein could be implemented as applications, scripts, plugins, or other function or software implemented within an EHR system, e.g., as modules or add-on features of an Epic™ EHR system. Integration with such an EHR system could also facilitate performance of aspects of the methods described herein, e.g., by using patient identity and rooming data to associate an activator with a particular patient by noting that the activator is already associated with a room and also noting that the particular patient has been assigned to the room.

To provide additional benefits, multiple timers can be maintained as described herein for a single patient, or for other subjects of a healthcare system (e.g., patient beds, patient rooms, operating rooms, dialysis machines, etc.). For example, four timers could be maintained for a single patient, with each timer related to a respective one of the “Four P's”: ‘Potty’ (assisting or otherwise attending to the patient's need to urinate/defecate, whether in their bed or by assisting the patient to move to a toilet), ‘Pain’ (assessing and/or taking steps to manage the patient's pain), ‘Personal Possessions” (making certain that the patient has access to their personal effects and/or assisting them in accessing or putting away such items), and “Positioning” (positioning/repositioning the patient, e.g., to reduce the incidence of pressure ulcers, to accommodate drainage or other post-surgical considerations). One or more of the timers for a patient, or for the patient's room/bed, could be related to regularly-performed tasks that are not performed by a healthcare professional and/or that are not direct patient care tasks, e.g., cleaning the patient's room, cleaning the patient's bathroom, providing social enrichment to the patient, testing the patient for a communicable disease (e.g., COVID-19), patrolling a patient's room or other location by a security guard, etc.

An activator as described herein could include multiple different buttons or other inputs (e.g., touch pads) to facilitate a healthcare professional or other staff (e.g., housekeeping, security, maintenance) to indicate which task has been performed and accordingly which timer associated with the patient/location to reset. For example, an activator could include four buttons, one for each of the “Four P's.” Additionally or alternatively, the identity or classification of the person activating the activator could be determined and used to select which timer to reset. For example, it could be determined that a nurse or other healthcare provider was located proximate to the activator (e.g., using an RTLS system based on a badge or other device worn/carried by the nurse) within a specified time period (e.g., +/−two minutes) of a time when the activator was activated a first time. In response, a first timer related to tasks performed by the nurse (e.g., a rounding task) could be reset in response to the activator being activated the first time. It could then be determined that another staff person (e.g., housekeeping staff, security staff) was located proximate to the activator within a specified time period of a time when the activator was activated a second time. In response, a second timer related to tasks performed by the other staff person (e.g., a housekeeping task, a security patrol task) could be reset in response to the activator being activated the second time.

Such different timers could be displayed in the same place (e.g., the same display at a nurse's station) or in different places, according to what tasks or classes of staff members are associated with the timers. For example, timers related to nurse rounding task(s) could be displayed at a nurse's station while timers related to housekeeping tasks could be displayed on a display located in a housekeeping department and/or on phones or tablets carried by housekeeping staff. Where the timers are related to different tasks that can be performed by the same type of staff (e.g., a first timer related to generic nurse rounding, and a second timer related specifically to repositioning a patient to avoid pressure sores), it can be beneficial to display the status of the timers in the same location. This can allow the staff to act in a more efficient manner by preemptively performing task(s) whose timers have not yet reached their thresholds at the same time as performing task(s) whose timers have reached their thresholds. This can also allow healthcare staff to reduce the total number of discrete interactions they have with different patients, thereby reducing the potential spread of airborne illnesses or other communicable diseases. Such multiple display could include selectively displaying multiple timers related to a single patient/location in order to balance the benefits of performing multiple tasks at once with the cost of performing some tasks at a rate greater than a specified base rate. For example, a second, sub-threshold timer for a patient could be displayed (e.g., on a nurse station display) along with a first, super-threshold timer for the patient if the second timer has reached more than a specified percentage of its threshold duration, e.g., if it has reached at least 75% of its threshold duration.

As noted above, the systems and methods described herein can help to improve the rate of performance of regularly repeating tasks (e.g., nurse rounding) by providing a display indicating which patients/tasks are due and also by providing a low-effort, privacy-preserving way for nurses or other staff to indicate that those tasks have been completed. These systems and methods also facilitate accurately monitoring, recording, and analyzing the timing and other aspects of the performance of such tasks. By providing highly accurate estimates of which patients require/are about to require the performance of such tasks, such systems and methods can also be used to more effectively allocate limited personnel resources to regions (e.g., floors, units) of a healthcare facility that are most in need of additional staff (e.g., available float nurse staff). This could include determining or adjusting a shift schedule, or re-allocating such staff in ‘real time’ across a shift as demands change. To do so, patients (and their associated timer(s)) can be organized into respective different geographical regions (e.g., floors of a facility, units within a facility, portions of floors of a facility). Timer data from each of the regions can then be analyzed, in real time or near real time, to determine aggregate levels of need or demand for staff in each of the regions. This could include determining a number of timers that have passed their respective threshold durations, a number of patients associated with at least one timer that has passed its threshold duration, an aggregate amount of time that all of the timers in a region have passed their respective threshold durations, or some other determination, based on the timers maintained for a region, of the overall level of staff demand that a region is experiencing. An indication of such determinations could then be transmitted to a display (e.g., a display at a nurse station, a display of a tablet or phone carried by an individual float nurse) to direct available staff to the region(s) where they are most needed. This could include providing the determined demand levels via the display (e.g., for the two or more highest-demand regions), providing an indication of one or more regions to proceed to (e.g., the highest-demand region(s)), or providing some other indication related to current demand for additional staff.

Note that throughout this disclosure the embodiments described herein are depicted as being used to facilitate regular performance of rounding activities by nurses or other healthcare professionals for patients in an inpatient healthcare setting. These depictions are not meant to be limiting, and are instead intended as an example application of the systems and methods described herein. It should be understood that these systems and methods may be applied to facilitate the repeated performance of a variety of different tasks in an inpatient or outpatient healthcare environment or in some other service environment. For example, the embodiments described herein could be used to facilitate regular recalibration or checking of medical equipment, e.g., to facilitate regular diagnostics or maintenance of dialysis machines, oxygen generators, infusion machines, hospital beds, or other equipment. In another example, the embodiments described herein could be used to facilitate regular checking and/or re-packing of bandages or other wound management interventions, regular inspection of wounds, regular wellness or state-of mind checks of intensive care patients, regular checks of vital signs and/or machinery status during a surgical intervention, or some other clinical intervention other than rounding that may be performed by nurses, doctors, anesthesiologists, or some other healthcare providers. Indications of timers or other reminders related to such tasks could be provided on the same displays via which rounding indications are provided (e.g., to alert nurses on a floor to imminent rounding needs as well as needs for bandage re-dressing or other repeated tasks) or may be provided via a different display (e.g., a reminder to perform recalibration, cleaning, and/or inspection of a dialysis machine could be provided on a tablet used by a technician).

Reference is made throughout this disclosure to the initialization, maintenance, pausing, or other operations relating to timers, and to providing displays related to such timers. Such timers could be set-duration and/or set-rate timers, such that the timers always increment at the same rate (e.g., one second per second) unless paused or stopped. However, such timers could have variable durations and/or variable rates in order to, e.g., measure the progression of a system or process. For example, a ‘timer’ could increment according to the flow rate of oxygen from an oxygen tank, according to the timing of administration of patient-controlled analgesics, or according to some other detected process or system. Such a the ‘timer’ could thus have the function of a meter of the underlying process, e.g., a meter indicating the level of use of a limited resource like the oxygen in an oxygen tank, or a meter indicating the rate of progression of a disease process, healing process, therapy process, or some other biological process. Such meter-type ‘timers,’ which may be referred to simply as ‘meters,’ may be used as the timer in any of the systems or methods described herein. Such embodiments could provide the benefits of the embodiments described herein to contexts where the timing of a healthcare provider's action (e.g., replacing an oxygen tank, replacing a reservoir of intravenous fluid, checking in on a patient's pain levels) depends not on a pre-specified duration or timeline but instead on the progression of a measurable quantity or process.

### II. Example Methods

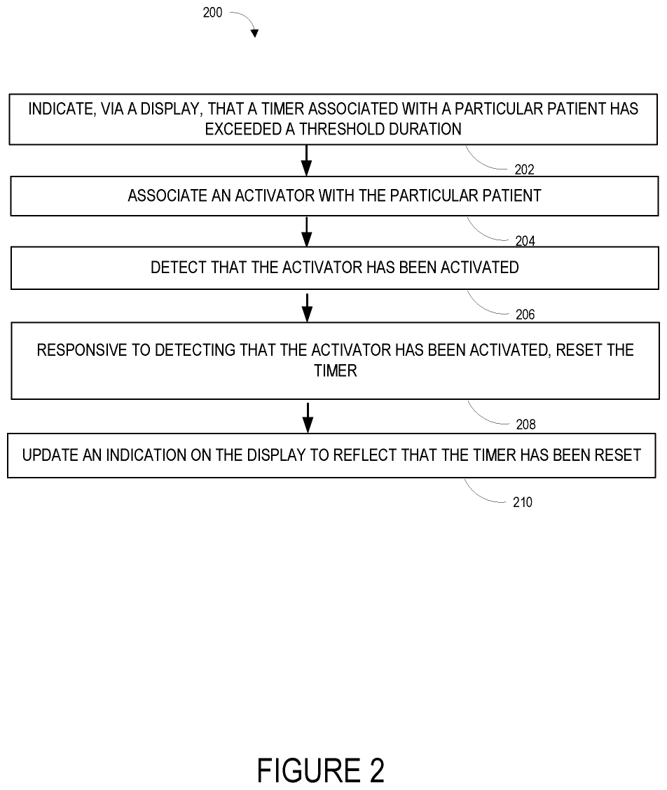

FIG. 2 illustrates an example method 200. The method 200 includes indicating, via a display, that a timer associated with a particular patient has exceeded a threshold duration (202). The method 200 additionally includes associating an activator with the particular patient (204). The method yet further includes detecting that the activator has been activated (206). The method 200 also includes, responsive to detecting that the activator has been activated, resetting the timer (208). The method 200 additionally includes updating an indication on the display to reflect that the timer has been reset. The method 200 could include additional or alternative elements or steps, e.g., as described elsewhere herein.

### III. Example Systems

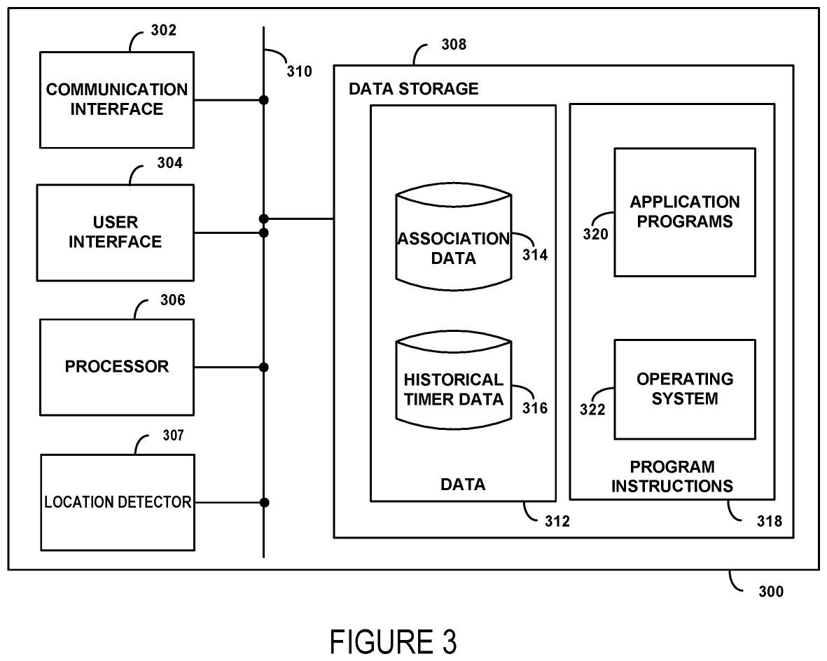

FIG. 3 illustrates an example computing device 300 that may be used to implement the methods described herein. By way of example and without limitation, computing device 300 may be a cellular mobile telephone (e.g., a smartphone), a computer (such as a desktop, notebook, tablet, or handheld computer, a server), elements of a cloud computing system, elements of an activator as described herein, elements of a display as described herein, or some other type of device. It should be understood that computing device 300 may represent a physical computing device such as a server, a particular physical hardware platform on which a timer, display, device location finding, patient association, patient recordkeeping, patient scheduling, or other application operates in software, or other combinations of hardware and software that are configured to carry out functions as described herein.

As shown in FIG. 3, computing device 300 may include a communication interface 302, a user interface 304, a processor 306, location detector 307, and data storage 308, all of which may be communicatively linked together by a system bus, network, or other connection mechanism 310. Such an example computing device 300 could include more of fewer of these elements. For example, the computing device 300 could be implemented as an activator as described elsewhere herein, in which case the user interface 304 of the computing device 300 could be limited to one or more buttons that could be pressed by a healthcare provider or other person to indicate that a rounding task or other regular task has occurred/been completed. In another example, the computing device 300 could be implemented as a display for displaying the status of one or more timers (associated with respective patients/activators) as described elsewhere herein, in which case the user interface 304 of the computing device 300 could be limited to a display for indicating such timer status information for a selection of patients (e.g., for patients roomed in a particular floor or other region with which the display is associated, e.g., as a display mounted above a nearby nurse's station). In yet another example, the computing device 300 could be implemented as a badge or other device worn by a nurse or other healthcare provider to facilitate locating the healthcare provider in real time (e.g., to determine that the healthcare provider is located proximate to an activator when the activator is activated) as described elsewhere herein, in which case the computing device 300 could lack a user interface 304 entirely, operating, among other things, to determine the location of the healthcare provider at one or more points in time and to report that location, via the communication interface 302), to a remote system.

Communication interface 302 may function to allow computing device 300 to communicate, using analog or digital modulation of electric, magnetic, electromagnetic, optical, or other signals, with other devices, access networks, and/or transport networks. Thus, communication interface 302 may facilitate circuit-switched and/or packet-switched communication, such as plain old telephone service (POTS) communication and/or Internet protocol (IP) or other packetized communication. For instance, communication interface 302 may include a chipset and antenna arranged for wireless communication with a radio access network or an access point. Also, communication interface 302 may take the form of or include a wireline interface, such as an Ethernet, Universal Serial Bus (USB), or High-Definition Multimedia Interface (HDMI) port. Communication interface 302 may also take the form of or include a wireless interface, such as a Wi-Fi, BLUETOOTH®, global positioning system (GPS), acoustical or radio-frequency real-time locating system (RTLS), or wide-area wireless interface (e.g., WiMAX or 3GPP Long-Term Evolution (LTE)). However, other forms of physical layer interfaces and other types of standard or proprietary communication protocols may be used over communication interface 302. Furthermore, communication interface 302 may comprise multiple physical communication interfaces (e.g., a Wi-Fi interface, a BLUETOOTH® interface, and a wide-area wireless interface).

In some embodiments, communication interface 302 may function to allow computing device 300 to communicate, with other devices, remote servers, access networks, and/or transport networks. For example, the communication interface 302 may function to transmit an indication that an activator has been activated to a remote server, to receive, from a remote server, an indication of one or more timers associated with respective patients, to transmit a determined location of an activator, patient, healthcare provider, or other person or object, or other information.

User interface 304 may function to allow computing device 300 to interact with a user, for example to receive input from and/or to provide output to the user. Thus, user interface 304 may include input components such as a keypad, keyboard, touch-sensitive or presence-sensitive panel, computer mouse, trackball, joystick, microphone, and so on. User interface 304 may also include one or more output components such as a display screen which, for example, may be combined with a presence-sensitive panel. The display screen may be based on CRT, LCD, and/or LED technologies, or other technologies now known or later developed. User interface 804 may also be configured to generate audible output(s), via a speaker, speaker jack, audio output port, audio output device, earphones, and/or other similar devices.

Location Detector 307 may include microphones, speakers or other sound emitters, radio frequency transmitters/receivers, light detectors/emitters, inertial measurement sensors/systems, or other elements configured to facilitate the determination of the absolute or relative location of the computing device 300. This could include communicating with, receiving signals (acoustical, RF, optical) emitted from, and/or emitting signals to an RTLS system. In some examples, the location detector 307 could include elements in common with and/or be a part of the communication interface 302, e.g., in examples wherein the transmission and/or reception of RF signals are used to determine the location of the computing device 300.

Processor 306 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). Data storage 308 may include one or more volatile and/or non-volatile storage components, such as magnetic, optical, flash, or organic storage, and may be integrated in whole or in part with processor 306. Data storage 308 may include removable and/or non-removable components.

Processor 306 may be capable of executing program instructions 318 (e.g., compiled or non-compiled program logic and/or machine code) stored in data storage 308 to carry out the various functions described herein. Therefore, data storage 308 may include a non-transitory computer-readable medium, having stored thereon program instructions that, upon execution by computing device 300, cause computing device 300 to carry out any of the methods, processes, or functions disclosed in this specification and/or the accompanying drawings or portions of such methods, processes, or functions. The execution of program instructions 318 by processor 306 may result in processor 306 using data 312.

By way of example, program instructions 318 may include an operating system 322 (e.g., an operating system kernel, device driver(s), and/or other modules) and one or more application programs 320 (e.g., functions for determining the absolute or relative locations of badges, activators, patients, healthcare providers, or other people or objects, maintaining one or more timers associated with each of a set of patients, beds, or locations, associating patients with activators or healthcare providers, polling timer or other patient data from a central server and displaying such information, and/or other methods as described herein) installed on computing device 300. Data 312 may include data about associations 314 between patients, activators, beds, badges, healthcare providers, or other people, objects, or locations, historical data 316 related to the running of timers (e.g., patient care rounding timers) as described elsewhere herein, or other data. Such data could be recorded in, compatible with, integrated into, or otherwise associated with an electronic health records (EHR) system, e.g., Epic™.

Application programs 320 may communicate with operating system 322 through one or more application programming interfaces (APIs). These APIs may facilitate, for instance, application programs 320 polling the status, location, or other information about badges, activators, beds, or other devices, associating such devices with patients, healthcare providers, beds, or other people, objects or locations and/or with each other, maintaining a number of timers associated with patients, activators, etc., collating statistics based on such information and related to the performance of patient care rounding tasks or other regular tasks, transmitting or receiving information via communication interface 302, receiving and/or displaying information on user interface 304, determining the location of the computing device 300, and so on.

Application programs 320 may take the form of “apps” that could be downloadable to computing device 300 through one or more online application stores or application markets (via, e.g., the communication interface 302). However, application programs can also be installed on computing device 300 in other ways, such as via a web browser or through a physical interface (e.g., a USB port) of the computing device 300. In some examples, the computing device 300 could be a server, elements of a cloud computing environment, or other system configured to operate as an electronic health records server (e.g., an Epic™ server). In such examples, the methods described herein for associating activators or other people/objects/locations with patients and/or healthcare providers, receiving or detecting the activation of such activators and using that information to run a set of timers associated with patients, recording information related to rounding tasks associated with the timers, or other functions could be implemented as application programs 320 that run on the electronic health records server. Such application programs 320 could rely on information and services provided by the electronic health records server (e.g., RTLS location information about badges, activators, patients, healthcare providers) and could provide healthcare-related information to the electronic health records server for storage and/or analysis (e.g., information on historical performance of patient care rounding tasks as detected using the methods described herein).

### IV. Enumerated Example Embodiments

Embodiments of the present disclosure may thus relate to one of the enumerated example embodiments (EEEs) listed below. It will be appreciated that features indicated with respect to one EEE can be combined with other EEEs.

EEE 1 is a method including: (i) indicating, via a display, that a timer associated with a particular patient has exceeded a threshold duration; (ii) associating an activator with the particular patient; (iii) detecting that the activator has been activated; (iv) responsive to detecting that the activator has been activated, resetting the timer; and (v) updating an indication on the display to reflect that the timer has been reset.

EEE 2 is the method of EEE 1, further including: (i) detecting a location of a nurse; (ii) detecting a location of the particular patient; and (iii) determining that the location of the nurse is proximate to the location of the particular patient, wherein resetting the timer responsive to detecting that the activator has been activated comprises resetting the timer responsive to detecting that the activator has been activated and that the location of the nurse is proximate to the location of the particular patient.

EEE 3 is the method of EEE 2, wherein determining that the location of the nurse is proximate to the location of the particular patient comprises determining that the location of the nurse is proximate to the location of the particular patient for more than a threshold period of time, wherein resetting the timer responsive to detecting that the activator has been activated comprises resetting the timer responsive to detecting that the activator has been activated and that the location of the nurse is proximate to the location of the particular patient for more than the threshold period of time.

EEE 4 is the method of any of EEEs 2-3 wherein detecting the location of the nurse comprises detecting, via a sensor proximate to the nurse, a first sound, infrared, or radio-frequency signal that is indicative of the location of the nurse, wherein detecting the location of the particular patient comprises detecting, via a sensor proximate to the particular patient, a second sound, infrared, or radio-frequency signal that is indicative of the location of the particular patient, and wherein determining that the location of the nurse is proximate to the location of the particular patient comprises determining that the first sound, infrared, or radio-frequency signal corresponds to the second sound, infrared, or radio-frequency signal.

EEE 5 is the method of any of EEEs 2-4, further including: recording, in a database, an indication of a timing of the activation of the activator, an identity of the nurse, and an identity of the particular patient.

EEE 6 is the method of any of EEEs 1-5, wherein indicating, via the display, that the timer associated with the particular patient has exceeded the threshold duration comprises providing an indication having a first color for the particular patient, and wherein updating the indication on the display to reflect that the timer has been reset comprises changing the indication from the first color to a second, different color.

EEE 7 is the method of EEEs 1-6, wherein indicating, via the display, that the timer associated with the particular patient has exceeded the threshold duration comprises providing an indication for the particular patient, and wherein updating the indication on the display to reflect that the timer has been reset comprises removing the indication from the display.

EEE 8 is the method of any of EEEs 1-6, wherein indicating, via the display, that the timer associated with the particular patient has exceeded the threshold duration comprises providing an indication having a first color for the particular patient, and wherein the method further included: determining that the timer has exceeded an additional, greater threshold duration and responsively changing the indication from the first color to a second, different color.

EEE 9 is the method of any of EEEs 1-8, wherein associating the activator with the particular patient includes: (i) associating the activator with a particular room; and (ii) determining that the particular patient is located proximate to the particular room.

EEE 10 is the method of EEE 9, wherein associating the activator with the particular room includes: (i) detecting, via a sensor that is part of a device that comprises the activator, a third sound, infrared, or radio-frequency signal that is indicative of the location of the activator; and (ii) determining that the third sound, infrared, or radio-frequency signal is indicative of the location of the particular room.

EEE 11 is the method of any of EEEs 9-10, wherein determining that the particular patient is located proximate to the particular room comprises accessing a database that contains a record that is indicative of at least one of the location of the particular patient or of a scheduled procedure of the particular patient.

EEE 12 is the method of any of EEEs 1-11, wherein associating the activator with the particular patient includes: (i) detecting a location of the particular patient; and (ii) determining that the location of the particular patient is proximate to a location of the activator, wherein resetting the timer responsive to detecting that the activator has been activated comprises resetting the timer responsive to detecting that the activator has been activated and that the location of the particular patient is proximate to the location of the activator.

EEE 13 is the method of EEE 12, wherein determining the location of the particular patient comprises detecting, via a patient sensor, a fourth sound, infrared, or radio-frequency signal that is indicative of the location of the patient sensor.

EEE 14 is the method of EEE 13, wherein associating the activator with the particular patient further includes: (i) detecting a location of the activator by detecting, via a sensor that is part of a device that comprises the activator, a fifth sound, infrared, or radio-frequency signal that is indicative of the location of the activator, wherein determining that the location of the particular patient is proximate to a location of the activator comprises determining that the fourth sound, infrared, or radio-frequency signal corresponds to the fifth sound, infrared, or radio-frequency signal.

EEE 15 is the method of any of EEEs 13-14, wherein the patient sensor is part of a device worn by the patient.

EEE 16 is the method of any of EEEs 13-14, wherein the patient sensor is disposed on or within a hospital bed.

EEE 17 is the method of any of EEEs 12-16, wherein resetting the timer responsive to detecting that the activator has been activated comprises resetting the timer responsive to detecting that the activator has been activated and that the location of the particular patient is proximate to the location of the activator.

EEE 18 is the method of any of EEES 12-17, further including: determining that the location of the particular patient is not proximate to the location of the activator and responsively performing at least one of resetting the timer or pausing the timer.

EEE 19 is the method of any of EEEs 1-18, further including: recording, in a database, an indication of a timing of the activation of the activator and an identifier for the particular patient.

EEE 20 is the method of any of EEEs 1-19, further including: accessing a database that contains a record that is indicative of an admission of the particular patient and responsively initiating the timer.

EEE 21 is the method of any of EEEs 1-10, wherein the activator comprises a button and wherein activating the activator comprises depressing the button.

EEE 22 is the method of any of EEEs 1-21, wherein the particular patient is located within a first region of a healthcare facility, and wherein the method further includes: (i) maintaining a first plurality of additional timers for additional patients located within the first region of the healthcare facility; (ii) maintaining a second plurality of additional timers for additional patients located within a second region of the healthcare facility; (iii) determining a first level of staff need for the first region based on the timer associated with the particular patient and the first plurality of additional timers; (iv) determining a second level of staff need for the second region based on the second plurality of additional timers; and (v) transmitting, to at least one of a user device or the display, an indication of at least one of the first level of staff need or the second level of staff need.

EEE 23 is the method of any of claims 1-22, wherein the timer associated with the particular patient is a first timer associated with the particular patient, wherein the threshold duration is a first threshold duration, wherein detecting that the activator has been activated comprises detecting that a first input of the activator has been activated, wherein resetting the timer responsive to detecting that the activator has been activated comprises resetting the first timer responsive to detecting that the first input of the activator has been activated, and wherein updating the indication on the display to reflect that the timer has been reset comprises updating a first indication on the display to reflect that the first timer has been reset, and wherein the method further includes: (i) indicating, via the display, that a second timer associated with the particular patient has exceeded a second threshold duration; (ii) detecting that a second input of the activator has been activated; (iii) responsive to detecting that the second input of the activator has been activated, resetting the second timer; and (iv) updating a second indication on the display to reflect that the second timer has been reset.

EEE 24 is the method of any of EEEs 1-32, wherein the timer associated with the particular patient is a first timer associated with the particular patient, wherein the threshold duration is a first threshold duration, wherein detecting that the activator has been activated comprises detecting that a first input of the activator has been activated, wherein resetting the timer responsive to detecting that the activator has been activated comprises resetting the first timer responsive to detecting that the first input of the activator has been activated, and wherein updating the indication on the display to reflect that the timer has been reset comprises updating a first indication on the display to reflect that the first timer has been reset, and wherein the method further includes: (i) indicating, via a second display, that a second timer associated with the particular patient has exceeded a second threshold duration; (ii) detecting that a second input of the activator has been activated; (iii) responsive to detecting that the second input of the activator has been activated, resetting the second timer; and (iv) updating a second indication on the second display to reflect that the second timer has been reset.

EEE 25 is the method of any of EEEs 23-24, wherein the first and second inputs of the activator are associated with respective first and second tasks selected from a set comprising: assisting or assessing the particular patient's need to urinate/defecate, assessing or treating the particular patient's pain, determining whether the particular patient has access to their personal effects, and positioning or repositioning the patient.

EEE 26 is the method of any of EEEs 1-22, wherein the timer associated with the particular patient is a first timer associated with the particular patient, wherein the threshold duration is a first threshold duration, wherein detecting that the activator has been activated comprises detecting that the activator has been activated at a first point in time, and wherein the method further includes: (i) detecting a location of a first staff person; (ii) determining that the location of the first staff person is proximate to a location of the activator within a time period that includes the first point in time, wherein resetting the timer responsive to detecting that the activator has been activated comprises resetting the first timer responsive to detecting that the activator has been activated at the first point in time and that the location of the first staff person is proximate to the location of the activator within a time period that includes the first point in time, and wherein updating the indication on the display to reflect that the timer has been reset comprises updating a first indication on the display to reflect that the first timer has been reset; (iii) indicating, via the display, that a second timer associated with the particular patient has exceeded a second threshold duration; (iv) detecting that the activator has been activated at a second point in time; (v) detecting a location of a second staff person; (vi) determining that the location of the second staff person is proximate to the location of the activator within a time period that includes the second point in time; (vii) responsive to detecting that the activator has been activated at the second point in time and that the location of the second staff person is proximate to the location of the activator within a time period that includes the second point in time, resetting the second timer; and (viii) updating a second indication on the display to reflect that the second timer has been reset.

EEE 27 is the method of any of EEEs 1-22, wherein the timer associated with the particular patient is a first timer associated with the particular patient, wherein the threshold duration is a first threshold duration, wherein detecting that the activator has been activated comprises detecting that the activator has been activated at a first point in time, and wherein the method further includes: (i) detecting a location of a first staff person; (ii) determining that the location of the first staff person is proximate to a location of the activator within a time period that includes the first point in time, wherein resetting the timer responsive to detecting that the activator has been activated comprises resetting the first timer responsive to detecting that the activator has been activated at the first point in time and that the location of the first staff person is proximate to the location of the activator within a time period that includes the first point in time, and wherein updating the indication on the display to reflect that the timer has been reset comprises updating a first indication on the display to reflect that the first timer has been reset; (iii) indicating, via a second display, that a second timer associated with the particular patient has exceeded a second threshold duration; (iv) detecting that the activator has been activated at a second point in time; (v) detecting a location of a second staff person; (vi) determining that the location of the second staff person is proximate to the location of the activator within a time period that includes the second point in time; (vii) responsive to detecting that the activator has been activated at the second point in time and that the location of the second staff person is proximate to the location of the activator within a time period that includes the second point in time, resetting the second timer; and (viii) updating a second indication on the second display to reflect that the second timer has been reset.

EEE 28 is the method of any of EEEs 26-27, wherein the first staff person is a healthcare provider, and wherein the second staff person is at least one of security personnel or housekeeping personnel.

EEE 29 is a system that includes: (i) an activator; (ii) a display; and (iii) a controller that includes one or more processors. The one or more processors are configured to perform controller operations that include the method of any of EEEs 1-28, performed using the activator and the display of the system.

EEE 30 is a non-transitory computer readable medium having stored therein instructions executable by a computing device to cause the computing device to perform the method of any of EEEs 1-28.

EEE 30 is a system including: (i) a controller comprising one or more processors; and (ii) a non-transitory computer readable medium having stored therein instructions executable by the controller device to cause the one or more processors to perform the method of any of EEEs 1-28.

### V. Conclusion

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid state drive, or another storage medium.

The computer readable medium can also include non-transitory computer readable media such as computer readable media that store data for short periods of time like register memory and processor cache. The computer readable media can further include non-transitory computer readable media that store program code and/or data for longer periods of time. Thus, the computer readable media may include secondary or persistent long term storage, like ROM, optical or magnetic disks, solid state drives, or compact-disc read only memory (CD-ROM), for example. The computer readable media can also be any other volatile or non-volatile storage systems. A computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims. As an example, the systems and methods described herein could be applied to applications and/or settings other than, or in addition to healthcare. For example, the systems and methods described herein could be applied to facilitate regular monitoring and management of security staff checking a variety of locations of a facility and/or performing one or more security-related tasks as such locations. In another example, the systems and methods described herein could be applied to facilitate regular maintenance of a set of machinery or other equipment and the monitoring of such maintenance, e.g., to clean or assess bioreactors at a facility for manufacturing antibodies or other biologics. In yet another example, the systems and methods described herein could be applied to facilitate regular checking, cleaning, or other tasks related to different aisles, product displays, or other elements or locations of a retail space and to facilitate the monitoring of performance of such tasks.

The systems and methods described herein can be advantageously applied to any situation where it is desirable to motivate, organize, and monitor the completion of a regularly-performed task while avoiding the effort and cost of paper logs and/or complex login sequences. The systems and methods described herein also have the benefit of preserving privacy/data security in that no information about the task (e.g., about the timing of performance of the task, about the identify or other information about a patient associated with the task) is readily available at the point of performance of the task, as the “activator” can be a simple button or other simple device that acts as a ‘dumb’ input to a remote system.

What is claimed is:

1. A system comprising:
an activator;
a display; and
a controller comprising one or more processors, wherein the one or more processors are configured to perform controller operations comprising:
indicating, via the display, that a timer associated with a particular patient has exceeded a threshold duration;
associating the activator with the particular patient;
detecting that the activator has been activated;
responsive to detecting that the activator has been activated, resetting the timer; and
updating an indication on the display to reflect that the timer has been reset.

2. The system of claim 1, wherein the controller operations further comprise:
detecting a location of a nurse;
detecting a location of the particular patient; and
determining that the location of the nurse is proximate to the location of the particular patient, wherein resetting the timer responsive to detecting that the activator has been activated comprises resetting the timer responsive to detecting that the activator has been activated and that the location of the nurse is proximate to the location of the particular patient.

3. The system of claim 2, wherein determining that the location of the nurse is proximate to the location of the particular patient comprises determining that the location of the nurse is proximate to the location of the particular patient for more than a threshold period of time, wherein resetting the timer responsive to detecting that the activator has been activated comprises resetting the timer responsive to detecting that the activator has been activated and that the location of the nurse is proximate to the location of the particular patient for more than the threshold period of time.

4. The system of claim 2, further comprising:
a first sensor, wherein detecting the location of the nurse comprises detecting, via the first sensor, a first sound, infrared, or radio-frequency signal that is indicative of the location of the nurse; and
a second sensor, wherein detecting the location of the particular patient comprises detecting, via the second sensor, a second sound, infrared, or radio-frequency signal that is indicative of the location of the particular patient, and wherein determining that the location of the nurse is proximate to the location of the particular patient comprises determining that the first sound, infrared, or radio-frequency signal corresponds to the second sound, infrared, or radio-frequency signal.

5. The system of claim 2, further comprising:
recording, in a database, an indication of a timing of the activation of the activator, an identity of the nurse, and an identity of the particular patient.

6-7. (canceled)

8. The system of claim 1, wherein indicating, via the display, that the timer associated with the particular patient has exceeded the threshold duration comprises providing an indication having a first color for the particular patient, and wherein the controller operations further comprise:
determining that the timer has exceeded an additional, greater threshold duration and responsively changing the indication from the first color to a second, different color.

9. The system of claim 1, wherein associating the activator with the particular patient comprises:
associating the activator with a particular room; and
determining that the particular patient is located proximate to the particular room.

10. The system of claim 9, further comprising:
a device that comprises the activator and an activator sensor, wherein associating the activator with the particular room comprises:
detecting, via the activator sensor, a third sound, infrared, or radio-frequency signal that is indicative of the location of the activator; and
determining that the third sound, infrared, or radio-frequency signal is indicative of the location of the particular room.

11. The system of claim 9, wherein determining that the particular patient is located proximate to the particular room comprises accessing a database that contains a record that is indicative of at least one of the location of the particular patient or of a scheduled procedure of the particular patient.

12. The system of claim 1, wherein associating the activator with the particular patient comprises:
detecting a location of the particular patient; and
determining that the location of the particular patient is proximate to a location of the activator, wherein resetting the timer responsive to detecting that the activator has been activated comprises resetting the timer responsive to detecting that the activator has been activated and that the location of the particular patient is proximate to the location of the activator.

13. The system of claim 12, further comprising:
a third sensor, wherein determining the location of the particular patient comprises detecting, via the third sensor, a fourth sound, infrared, or radio-frequency signal that is indicative of the location of the patient sensor.

14. The system of claim 13, further comprising:
a device that comprises the activator and an activator sensor, wherein associating the activator with the particular patient further comprises:
detecting a location of the activator by detecting, via the activator sensor, a fifth sound, infrared, or radio-frequency signal that is indicative of the location of the activator, wherein determining that the location of the particular patient is proximate to a location of the activator comprises determining that the fourth sound, infrared, or radio-frequency signal corresponds to the fifth sound, infrared, or radio-frequency signal.

15. (canceled)

16. The system of claim 13, wherein the third sensor is part of or configured to be mounted on or within a hospital bed.

17. The system of claim 12, wherein resetting the timer responsive to detecting that the activator has been activated comprises resetting the timer responsive to detecting that the activator has been activated and that the location of the particular patient is proximate to the location of the activator.

18. The system of claim 12, wherein the controller operations further comprise:
determining that the location of the particular patient is not proximate to the location of the activator and responsively performing at least one of resetting the timer or pausing the timer.

19-21. (canceled)

22. The system of claim 1, wherein the particular patient is located within a first region of a healthcare facility, and wherein the controller operations further comprise:
maintaining a first plurality of additional timers for additional patients located within the first region of the healthcare facility;
maintaining a second plurality of additional timers for additional patients located within a second region of the healthcare facility;
determining a first level of staff need for the first region based on the timer associated with the particular patient and the first plurality of additional timers;
determining a second level of staff need for the second region based on the second plurality of additional timers; and
transmitting, to at least one of a user device or the display, an indication of at least one of the first level of staff need or the second level of staff need.

23. The system of claim 1, wherein the timer associated with the particular patient is a first timer associated with the particular patient, wherein the threshold duration is a first threshold duration, wherein detecting that the activator has been activated comprises detecting that a first input of the activator has been activated, wherein resetting the timer responsive to detecting that the activator has been activated comprises resetting the first timer responsive to detecting that the first input of the activator has been activated, and wherein updating the indication on the display to reflect that the timer has been reset comprises updating a first indication on the display to reflect that the first timer has been reset, and wherein the controller operations further comprise:
indicating, via the display, that a second timer associated with the particular patient has exceeded a second threshold duration;
detecting that a second input of the activator has been activated;
responsive to detecting that the second input of the activator has been activated, resetting the second timer; and
updating a second indication on the display to reflect that the second timer has been reset.

24. The system of claim 1, wherein the timer associated with the particular patient is a first timer associated with the particular patient, wherein the threshold duration is a first threshold duration, wherein detecting that the activator has been activated comprises detecting that a first input of the activator has been activated, wherein resetting the timer responsive to detecting that the activator has been activated comprises resetting the first timer responsive to detecting that the first input of the activator has been activated, and wherein updating the indication on the display to reflect that the timer has been reset comprises updating a first indication on the display to reflect that the first timer has been reset, and wherein the controller operations further comprise:
indicating, via a second display, that a second timer associated with the particular patient has exceeded a second threshold duration;
detecting that a second input of the activator has been activated;
responsive to detecting that the second input of the activator has been activated, resetting the second timer; and
updating a second indication on the second display to reflect that the second timer has been reset.

25. (canceled)

26. The system of claim 1, wherein the timer associated with the particular patient is a first timer associated with the particular patient, wherein the threshold duration is a first threshold duration, wherein detecting that the activator has been activated comprises detecting that the activator has been activated at a first point in time, and wherein the controller operations further comprise:
detecting a location of a first staff person;
determining that the location of the first staff person is proximate to a location of the activator within a time period that includes the first point in time, wherein resetting the timer responsive to detecting that the activator has been activated comprises resetting the first timer responsive to detecting that the activator has been activated at the first point in time and that the location of the first staff person is proximate to the location of the activator within a time period that includes the first point in time, and wherein updating the indication on the display to reflect that the timer has been reset comprises updating a first indication on the display to reflect that the first timer has been reset;
indicating, via the display, that a second timer associated with the particular patient has exceeded a second threshold duration;
detecting that the activator has been activated at a second point in time;
detecting a location of a second staff person;
determining that the location of the second staff person is proximate to the location of the activator within a time period that includes the second point in time;
responsive to detecting that the activator has been activated at the second point in time and that the location of the second staff person is proximate to the location of the activator within a time period that includes the second point in time, resetting the second timer; and
updating a second indication on the display to reflect that the second timer has been reset.

27. The system of claim 1, wherein the timer associated with the particular patient is a first timer associated with the particular patient, wherein the threshold duration is a first threshold duration, wherein detecting that the activator has been activated comprises detecting that the activator has been activated at a first point in time, and wherein the controller operations further comprise:
detecting a location of a first staff person;
determining that the location of the first staff person is proximate to a location of the activator within a time period that includes the first point in time, wherein resetting the timer responsive to detecting that the activator has been activated comprises resetting the first timer responsive to detecting that the activator has been activated at the first point in time and that the location of the first staff person is proximate to the location of the activator within a time period that includes the first point in time, and wherein updating the indication on the display to reflect that the timer has been reset comprises updating a first indication on the display to reflect that the first timer has been reset;
indicating, via a second display, that a second timer associated with the particular patient has exceeded a second threshold duration;
detecting that the activator has been activated at a second point in time;
detecting a location of a second staff person;
determining that the location of the second staff person is proximate to the location of the activator within a time period that includes the second point in time;
responsive to detecting that the activator has been activated at the second point in time and that the location of the second staff person is proximate to the location of the activator within a time period that includes the second point in time, resetting the second timer; and
updating a second indication on the second display to reflect that the second timer has been reset.

28-58. (canceled)
